SEQUENCE LISTING (7 sequences)

SEQ ID NO: 1 - DNA, 685 bp, Pholiota Glutinosior
tcctccgcttattgatatgcttaagttcagcgggtagtcctacctgatttgaggtcaaattgtcatatattgtccgaagacgattagaagcggtgctactatggcagttagtcaatggcgtagataattatcacaccaatagacagttcgcgcgagcgaccagctaatgcattttaggggagaaggctcgcgagaaaccttcaaaaaattttcccccccacatccaagtctttattaacaaaagctaataagattgagaatttaatgacactcaaacaggcatgctcctcggaataccaaggagcgcaaggtgcgttcaaagattcgatgattcactgaattctgcaattcacattacttatcgcatttcgctgcgttcttcatcgatgcgagagccaagagatccgttgctgaaagttgtatatagtttataggtaacaagacccattattacattctgttacatttttatggtgtatatgaaaacatagatctgaaattacaggcaaagccgcttcgcagcagcattccccactactgagttgcctcataaatttattccagacctacaatgtgtgcacaggtggagagataaagatggcagacgtgcacatgcccagatgggccagcaacaatcataccaagttcattcaataatgatccttccgcaggttcacctacggaa

SEQ ID NO: 2 - DNA, 684 bp, Pholiota Glutinosior
tcctccgcttattgatatgcttaagttcagcgggtagtcctacctgatttgaggtcaaattgtcatatattgtccgaagacgattagaagcggtgctactatggcagttagtcaatggcgtagataattatcacaccaatagacagttcgcgcgagcgaccagctaatgcattttaggggagaaggctcgcgagaaaccttcaaaaaattttcccccccacatccaagtctttattaacaaaagctaataagattgagaatttaatgacactcaaacaggcatgctcctcggaataccaaggagcgcaaggtgcgttcaaagattcgatgattcactgaattctgcaattcacattacttatcgcatttcgctgcgttcttcatcgatgcgagagccaagagatccgttgctgaaagttgtatatagtttataggtacaagacccattattacattctgttacatttttatggtgtatatgaaaacatagatctgaaattacaggcaaagccgcttcgcagcagcattccccactactgagttgcctcataaatttattccagacctacaatgtgtgcacaggtggagagataaagatggcagacgtgcacatgcccaaatgggccagcaacaatcataccaagttcattcaataatgatccttccgcaggttcacctacggaa

SEQ ID NO: 3 - DNA, 685 bp, Pholiota Glutinosior
tcctccgcttattgatatgcttaagttcagcgggtagtcctacctgatttgaggtcaaattgtcatatattgtccgaagacgattagaagcggtgctactatggcagttagtcaatggcgtagataattatcacaccaatagacagttcgcgcgagcgaccagctaatgcattttaggggagaaggctcgcgagaaaccttcaaaaaattttccccccccacatccaagtctttattaacaaaagctaataagattgagaatttaatgacactcaaacaggcatgctcctcggaataccaaggagcgcaaggtgcgttcaaagattcgatgattcactgaattctgcaattcacattacttatcgcatttcgctgcgttcttcatcgatgcgagagccaagagatccgttgctgaaagttgtatatagtttataggtacaagacccattattacattctgttacatttttatggtgtatatgaagacatagatctgaaattacaggcaaagccgctttgcagcagcattccccactactgagttacctcagaaatttattccagacctacaatatgtgcacaggtggagagataaagatggcagacgtgcacatgcccagatgggccagcaacaatcataccaagttcattcaataatgatccttccgcaggttcacctacggaa

SEQ ID NO: 4 - DNA, 1233 bp, Pholiota Glutinosior
tggccatccgagttgtaatctagagaagtgttatccgcgctggaccgtgtacaagtctcctggaatggagcgtcatagagggtgagaatcccgtctttgatacggactgccagggctttgtgatgcgctctcaaagagtcgagttgtttgggaatgcagctcaaaatgggtggtaaattccatctaaagctaaatattggcgagagaccgatagcgaacaagtaccgtgagggaaagatgaaaagaactttggaaagagagttaaacagtacgtgaaattgctgaaagggaaacgcttgaagtcagtcgcgttgtctggggatcaaccttacttttgttgggcgtactttccagatgacgggtcagcatcaattttgaccgttggataaagtctagaggaatgtggcatcttcggatgtgttatagcctttggtcgcatacaacggttgggattgaggaactcagcacgccgaaaggccgggtacttgtaccacgttcgtgcttaggatgctggcataatggctttaatcgacccgtcttgaaacacggaccaaggagtctaacatgcctgcgagtatttgggtggaaaacccgagtgcgtaatgaaagtgaaagttgagatccctgtcatggggagcatcgacgcccggaccagaccttttgtgacggatctgcggtagagcatgtatgttgggacccgaaagatggtgaactatgcctgaatagggtgaagccagaggaaactctggtggaggctcgtagcgattctgacgtgcaaatcgatcgtcaaatttgggtataggggcgaaagactaatcgaaccatctagtagctggttcctgccgaagtttccctcaggatagcagaaactcatttcagatttatgtggtaaagcgaatgattagaggccttggggttgaaacaaccttaacctattctcaaactttaaatatgtaagaacaaaccgtctcttgattggaccgtttggcgattgagagtttctagtgggccatttttggtaagcagaactggcgatgcgggatgaaccgaacgcgaggttaaggtgccggaattcacgctcatcagacaccacaaaaggtgttagttcatctagacagcaggacggtggccatggaagtcggaatccgctaaggagtgtgtaacaactcacctgccgaatgaactagccctgaaaatggatggcgcttaagcgtgatacccatacctcgccgt

SEQ ID NO: 5 - DNA, 883 bp, Pholiota Glutinosior
gcctcgtcaactgcataatacgcattggggaatggtctgtcccgccgaaaccccagaaggtcaagcttgcggtcttgtcaaaaatttggcgctcatgtcatgtatttccgttggatctccatctgcacccgtcatcgaattcctggaggagtggggtttggaatctttggaagaaaatgcccattcgacaacaccttgtactaaggtgttcgtaaacggtgtgtggatgggtgtgcatcgcgatgctgccaacctggtcaagacgttgaagaaattgcggaggagagatgatatcagtcctgaagtttccgtcgtgcgagatattcgagaaaaggaactgcggctatatactgatgcaggacgcgtctgccgaccgctgttcatcgtggagaatcagcaacttgcgctccagaagaagcacattagatggctcagcaacggttataatgaggaaggagaggaattcaagtgggaacatctgatcaagggcggtgttgtcgagctattggatgctgaggaagaagagacagtcatgatttctatgactcccgaagatctggagaactctcggctccagcaaagtggtgttgttcctcctcctaatgacggagagtttgatcctgctgcccgacttaaagctgggacacatgcacatacttggacgcattgtgaaattcatccgagcatgattctcggcatttgtgccagtatcattcctttcccggatcacaatcaagtaggttcatttatgatcatactcatcctcaatactcattattttatcagtctcctcgtaacacataccaatctgccatgggcaagcaagccatgggaatcttccttaccaattttctgattcgcatggacaccatggccaaacatc

SEQ ID NO: 6 - DNA, 868 bp, Pholiota Glutinosior
tcgtcaactgcataatacgcattggggaatggtctgtcccgccgaaaccccagaaggtcaagcttgcggtcttgtcaaaaatttggcgctcatgtcatgtatttccgttggatctccatctgcacccgtcatcgaattcctggaggagtggggtttggaatctttggaagaaaatgcccattcgacaacaccttgtactaaggtgttcgtaaacggtgtgtggatgggtgtgcatcgcgatgctgccaacctggtcaagacgttgaagaaattgcggaggagagatgatatcagtcctgaagtttccgtcgtgcgagatattcgagaaaaggaactgcggctatatactgatgcaggacgcgtctgccgaccgctgttcatcgtggagaatcagcaacttgcgctccagaagaagcacattagatggctcagcaacggttataatgaggaaggagaggaattcaagtgggaacatctgatcaagggcggtgttgtcgagctattggatgctgaggaagaagagacagtcatgatttctatgactcccgaagatctggagaactctcggctccagcaaagtggtgttgttcctcctcctaatgacggagagtttgatcctgctgcccgacttaaagctgggacacatgcacatacttggacgcattgtgaaattcatccgagcatgattctcggcatttgtgccagtatcattcctttcccggatcacaatcaagtaggttcatttatgatcatactcatcctcaatactcattattttatcagtctcctcgtaacacataccaatctgccatgggcaagcaagccatgggaatcttccttaccaattttctgattcgcatggacacca

SEQ ID NO: 7 - DNA, 866 bp, Pholiota Glutinosior
cgtcaactgcataatacgcattggggaatggtctgtcccgccgaaaccccagaaggtcaagcttgcggtcttgtcaaaaatttggcgctcatgtcatgtatttccgttggatctccatctgcacccgtcatcgaattcctggaggagtggggtttggaatctttggaagaaaatgcccattcgacaacaccttgtactaaggtgttcgtaaacggtgtgtggatgggtgtgcatcgcgatgctgccaacctggtcaagacgttgaagaaattgcggaggagagatgatatcagtcctgaagtttccgtcgtgcgagatattcgagaaaaggaactgcggctatatactgatgcaggacgcgtctgccgaccgctgttcatcgtggagaatcagcaacttgcgctccagaagaagcacattagatggctcagcaacggttataatgaggaaggagaggaattcaagtgggaacatctgatcaagggcggtgttgtcgagctattggatgctgaggaagaagagacagtcatgatttctatgactcccgaagatctggagaactctcggctccagcaaagtggtgttgttcctcctcctaatgacggagagtttgatcctgctgcccgacttaaagctgggacacatgcacatacttggacgcattgtgaaattcatccgagcatgattctcggcatttgtgccagtatcattcctttcccggatcacaatcaagtaggttcatttatgatcatactcatcctcaatactcattattttatcagtctcctcgtaacacataccaatctgccatgggcaagcaagccatgggaatcttccttaccaattttctgattcgcatggacacc

(19) United States
(12) Patent Application Publication
(10) Pub. No.: US 2021/0130767 A1
(43) Pub. Date: May 6, 2021

(54) Species of Genus Pholiota and Artificial Cultivation Method and Use Thereof

(71) Applicants: Guangdong Institute of Microbiology (Guangdong Detection Center of Microbiology), Guangzhou (CN); Guangdong Yuewei Edible Fungi Technology Co., Ltd., Guangzhou (CN)

(72) Inventors: Qingping Wu, Guangzhou (CN); Huiping Hu, Guangzhou (CN); Yuanchao Liu, Guangzhou (CN); Xiaowei Liang, Guangzhou (CN); Yizhen Xie, Guangzhou (CN); Zhi Zhang, Guangzhou (CN)

(73) Assignees: Guangdong Yuewei Edible Fungi Technology Co., Ltd.; Guangdong Institute of Microbiology (Guangdong Detection Center of Microbiology)

(21) Appl. No.: 16/832,561

(22) Filed: Mar. 27, 2020

(30) Foreign Application Priority Data
Nov. 1, 2019 (CN) 201911057539.X

(51) Int. Cl. C12N 1/14; A61K 36/07; C12R 1/645; A23L 31/00; A23L 33/00

(52) U.S. Cl. CPC C12N 1/14; A61K 36/07; A23V 2002/00; A23L 31/00; A23L 33/40; C12R 1/645

(57) ABSTRACT

The invention relates to a rare edible fungus new strain and an artificial cultivation method and use thereof, in particular to a new species Pholiota glutinosior of genus Pholota and its artificial cultivation method and use. The new strain of genus Pholiota of the present invention is collected from the Guangdong Chebaling National Nature Reserve, and is identified as a new strain of genus Pholiota, and the original strain is obtained by tissue separation, and is named as HMGIM-W140054, deposited on Jun. 3, 2019, at China Center for Type Culture Collection (CCTCC, Wuhan, China), with the accession number CCTCC NO: M 2019414. The new strain of the invention has been artificially domesticated and cultivated, exhibits a strong and significant inhibition rate against Staphylococcus, and has a high content of histidine, crude polysaccharide and potassium. It is rich in nutrients and has high cultivation yield, and is a new species with high development prospects.

### REFERENCE TO SEQUENCE LISTING SUBMITTED VIA EFS-WEB

This application references sequence listings contained in an ASCII text file named H-003P006 USYW1912 Sequences, created on May 28, 2020, which is 8.75 kb in size and electronically submitted via EFS-Web herewith, which are incorporated herein by reference in its entirety.

### TECHNICAL FIELD

The application relates to a new species of rare edible fungus and an artificial cultivation method and use thereof, in particular to a new species of genus Pholiota and an artificial cultivation method and use thereof.

### BACKGROUND

At present, the industry of edible and medicinal fungi has developed rapidly. According to the statistics of China Edible Fungus Association, the output of edible and medicinal fungi in China reached 37.12 million tons in 2017, increased by 3.21% compared with 2016, and the output value was 272.192 billion. China's output value accounts for more than 75% of the world, and employs more than 20 million people. The edible fungus industry ranks fifth in the planting industry behind food, vegetables, fruits and oil, exceeding tea and sericulture.

Now, with the flourishing development of the edible and medicinal fungi industry, more and more species of rare edible and medicinal fungi have been gradually discovered and applied. Many of the original rare varieties have been domesticated, such as Dictyophora, Tea tree mushroom, Pleated umbrella and Morel and so on. However, a large number of wild edible medicinal fungi have not been studied because of they have not been recognized.

According to research, there are currently more than 3 million species of fungi in the world, only 1% of which are known, about 14,000 species of fungi are known as large fungi, and 1789 kinds of edible fungi in China and 798 kinds of medicinal fungi are domestically recognized. Among them, less than 100 kinds of wild edible and medicinal fungi have been domesticated, and there are only more than 30 kinds varieties of large-scale cultivation. There is still a long way before humans can study and utilize large fungi. With the gradual increase of people's living standards, the requirements for quality of life are higher, and large fungi are very rich in human health because they are rich in various nutrients and functional ingredients, including fungal polysaccharides, triterpenoids, and sterols and so on. Large fungi are getting more and more attention.

Pholiota belongs to Basidiomycota, Agaricomycotina, Agaricus, Strophariaceae. Pholiota was established in 1871 by Kummer, and the type species is Pholiota squarrosa (Fr.) Kumm. Strophariaceae was established in 1927 by Overeem. In 1946, Singer & Smith published eight genera including the Pholiota, and the classification of the family was controversial, but Pholiota always belonged to an independent genus or subfamily.

In 1871, Kummer raised the two families of Trib. Pholiota (including sixteen species) and Tribe. Flammula (including fifteen species) established by Fries in 1821 to the genus, namely Pholiota (Fr.) P. Kumm. and Flammula (Fr.) P. Kumm. At that time, Fries thought that the difference between Trib. Pholiota and Tribe. Flammula was the texture of the fungus ring and the ease of falling off. However, in 1874, he changed this statement and believed that there was no obvious boundary between them. In 1886, Quelet suggested that these two families be classified as subgenus in the genus Dryophila, namely Subgen. Pholiota and Subgen. Flammula. Since then, although at the same time the taxonomists have been arguing over the taxonomic status of these two families, most people still support Kummer's point of view, so this view gradually dominates. Singer and Smith (1946) analyzed the reasons for this divergence, they thought that the taxonomists were arguing because the difference between Pholiota (Fr.) P. Kumm and Flammula (Fr.) P. Kumm. was too small and there was no more sufficient evidence to support a certain point of view. Singer tried to distinguish the two groups with the phylogenetic tree. His concept was undoubtedly a positive promotion for the development of fungus classification, and many subsequent authors adopted his system classification.

The classification system in the genus Pholiota has also been a matter of debate among fungal taxonomists. Different taxonomists have proposed different schemes. Singer (1963) analyzed the entire population with detail data, and classify the genus Pholiota into 3 subgroups 12 groups. Smith and Hesler (1968) argued that Singer's typical species research methods did not fully and reasonably reflect this group, and this method cannot conduct comparative studies on larger groups, so it is difficult to cope with larger genus in detail. In view of this point, they put forward their own point of view, that is, all the species with the characteristics of “The spore print is rusted brown or yellowish brown, the spore is smooth, and the top has bud germ pores, and the inner veil is present on the stipe flocculently” are classified in the genus Pholiota, there are 7 subgenus 16 section and 48 series. Bas (1971) disagreed with Smith and Hesler's argument, and considered that they limited the scope of the genus too broadly, and the wider the range, the greater the change in characteristics, thereby separating the genus associated with genus Pholiota and its relatively unimportant genus. This is not conducive to the development of the classification of genus. Singer (1975) refuted Smith and Hesler's views on the classification system, arguing that it is not a natural classification of genus, and advocated the classification of the family Strophariaceae into a number of genus, thus making the phylogenetic relationship clearer. Therefore, he further revised the classification system of the genus Pholiota and classified into 5 subgenus 14 section and 25 series. Hawksworth et al. (1983, 1995) used the classification system of Singer (1975). Jacobsson (1990) also believes that the scope of the Smith and Hesler classification systems is too wide, and it is easy to classify the species of other genus into the genus Pholiota, so they do not agree with their views and propose the classification system of 6 subgenus 13 section. Kirk et al. (2001, 2008) used the classification system of Jacobsson (1990). Smith (1979) still insists on his own point of view in his article, emphasized that the scope of the genus does not affect the relationship of phylogeny.

Regarding the classification on basis of subgenus and similar genus, many taxonomists have put forward their own views. Smith and Hesler (1968) considered that the color and surface characteristics of the cap, the color of the flesh and lamellae, the surface characteristics of the stipe, and the characteristics of the veil are important macroscopic features of genus Pholiota; and the size and the shape of the spores, the state of the spores wall, the presence and the type of the lateral cystidium, the epidermal structure of the cap and the presence or absence of the stylocystidium are important microscopic features of genus Pholiota. Singer (1975) considered that these characteristics including the change of spore wall in Melzer's reagent, the structure of the spore tip, the surface texture, the color of the spore print, the gelation of the cystidium, the hymenium and the upper epidermis hyphae can be used as important classification basis under genus Pholiota. Pegler (1971) emphasizes that only the spore wall state, the exact color of the spore print, and the changes in the spore tip observed under different conditions can better distinguish the species of genus Pholiota. Jacobsson (1990) discusses the difference between genus Pholiota and other genus of the family Strophariaceae. It is believed that the color of the spore print and the nature of the spore wall are their main differences.

At present, China Flora Fungorum Sinicorum—Strophariaceae (2014) records 56 species of genus Pholiota in China, and Atlas of Chinese Macrofungal Resouces (2015) contained 16 species of genus Pholiota. Many varieties have found the same name and have been revised after continuous development of molecular biology.

Many species of genus Pholiota, such as P. microspora, P. adiposa, etc., which are famous edible fungi, and are delicious and have been produced on a large scale. Hui Fengli et al. (2003) pointed out that the content of minerals in the fruit body of Padiposa is higher than that of the general edible fungi. In addition, genus Pholiota has a strong ability to adsorb and accumulate mineral elements. Nishimoto and Fujita (1977) pointed out that the content of Cd in the sleepers of genus P. microspora was positively correlated with the content in the fruiting bodies. Kitanovic et al. (2001) showed that woody fungi such as Pholiota squarrosa tend to adsorb mineral elements such as K, Fe and Cu. The fruit body of P. microspora has strong accumulation ability for minerals K, Na, P, Mg, Cu, Zn, Cd, Ca, etc. in the culture material.

Yang Shanshan (1988) reported that the vitamin content per 100 g of P. microspora was 0.05 mg vitamin B2, 8.83 mg vitamin C, and 0.223 mg vitamin D. Hui Fengli et al (2003) identified that P. adiposa contained vitamins including vitamin B1, vitamin B2 and nicotinamide (VPP), in which vitamin B2 is 1.5 mg/100 g, which is higher than that of common medicinal fungi (1.229 mg/100 g), moreover, it contained a large amount of ergosterol (precursor of vitamin D2). Hui Fengli et al. (2003) proved that there are many kinds of amino acid (18) in the fruit body of P. adiposa, and the total content is higher than other edible medicinal fungi; the essential amino acid composition is reasonable by the equilibrium model of essential amino acids. Zhao Zhanguo and Yang Xiulan (1985) and Su Yanyou and Gao Lijun (2003) respectively determined the sixteen kinds of and seventeen kinds of amino acid contents of fruiting bodies, and they all obtained similar results. On basis of this research, Su Yanyou (2002) concluded that the amino acid content in P. adiposa is slightly lower than that in mushroom, but much higher than that in Hericium, and the essential amino acid content (six kinds of amino acid) is mostly higher than the latter two. Mitsuaki (1967) studied the mucus composition of P. microspora: 74.2% carbohydrate, in which 57.9% glutamic acid, 6.5% xylan, and a small amount of sucrose, galactose and arabinose, ash was 18.6%. Yang Shanshan (1988) determined the contents of crude protein, pure protein, fat and total sugar in P. microspora, and obtained 33.76 g, 15.13 g, 4.03 g and 38.99 g per 100 g of dry product respectively. Rui Shihua (2001) also reported that the content of fresh products per 100 g of P. microspora was 1.1 g protein, 0.2 g fat and 2.5 g carbohydrate.

Huang Nianlai (1998) reported that the inhibitory rate of hot water extract (polysaccharide) of P. microspora on mouse sarcoma 180 is 86.5%. P. adiposa fruit body polysaccharides on mouse sarcoma 180 and Ehrlich ascites carcinoma is 80%-90%. In addition, the two can prevent infection by Staphylococcus, Escherichia coli, and Klebsiella pneumoniae. Yang Shanshan (1988) pointed out that the mucus on the surface of P. microspora and P. adiposa fruit body helps the recovery of body and brain power. Su Yanyou et al. (2004) demonstrated that P. adiposa polysaccharides have the function of immunopotentiators, which can effectively activate macrophages, regulate the immune system by multiple ways such as enhancing cytokine secretion, enhancing NO production, enhancing phagocytic function and killing activity in vitro. Sato and Yoshida (1979) extracted oily syrup (HM-32) from Pholiota terrestris, which increases the viability of mouse spleen cells and lymphocytes. Coulet and Guillot (1972) extracted human erythrocyte lectin from Pholiota squarrosa and proposed its adhesion point on blood cells. Kawagishi et al. (1991) isolated lectin PAA from the fruit body of Pholiota autivella, and measured its molecular mass and N-terminal amino acid sequence. Furukawa et al. (1995) extracted H-type hemagglutinin from Pholiota squarrosa, which helped to agglutinate human 0-type blood cells. In addition, Ikekawa et al. (2001) extracted anti-tumor, anti-oxidation EEM-S substance from P. microspora that can enhance immunity and suppresses blood pressure and lower blood sugar. Badalyan (2003) demonstrated that Pholiota alnicola has antioxidant activity (AOA), which inhibits peroxide oxidation in the brain of mice, with AOA >20%.

Moreover, most species of genus Pholiota can be eaten. It has strong ability to decompose lignin, cellulose and hemicellulose, and it produces high-quality, high-protein and low-fat food with delicious taste and fruit body. It is a potential edible cultivar.

According to the classification of genus Pholiota and its nutrition and function, the species with transformation potential among the genus deserve our attention.

### SUMMARY OF THE INVENTION

In response to the above deficiencies, the present invention provides a novel fungus species Pholiota glutinosior, which is a wild rare edible fungus, and its artificial domestication and use.

The present invention achieves the object by the following scheme:

In the first aspect, the new strain of genus Pholiota of the present invention is collected from the Guangdong Chebaling National Nature Reserve, and is identified as a new strain of genus Pholiota, and the original strain is obtained by tissue separation, and is named as HMGIM-W140054, deposited on Jun. 3, 2019, at China Center for Type Culture Collection (CCTCC, Wuhan, China), with the accession number CCTCC NO: M 2019414.

In the second aspect, the present invention provides an artificial cultivation method of Pholiota glutinosior CCTCC NO: M 2019414, which comprises producing the first-class strain, producing the second-class strain, producing the third-class strain, cultivating culture and cultivation management, by weight percent, the cultivating medium comprises 28-32% cottonseed hulls, 56-58% wood chips, 8-12% bran, and 1-2% CaCO₃.

In the third aspect, the present invention provides the use of a new species Pholiota glutinosior CCTCC NO: M 2019414 or an extract thereof for use in diseases associated with anti-bacteria.

In the fourth aspect, the present invention provides the use of a new specie Pholiota glutinosior CCTCC NO: M 2019414 or an extract thereof for preparation of the medicament for treating diseases caused by anti-bacteria or for preparation of the health supplement.

In the fifth aspect, the present invention provides the medicament for treating diseases caused by anti-bacteria, comprising a new species Pholiota glutinosior CCTCC NO: M 2019414 or an extract thereof and a carrier.

In the sixth aspect, the present invention provides the health care product comprising a new specie Pholiota glutinosior CCTCC NO: M 2019414 or an extract thereof.

The new strain of the invention has been artificially domesticated and cultivated, exhibits strong and significant inhibition rate against Staphylococcus, and has high content of histidine, crude polysaccharide and potassium. It is rich in nutrients and has high cultivation yield, and is a new species with high development prospects.

### BRIEF DESCRIPTION OF DRAWINGS

FIG. 1 is wild fruiting body of Pholiota glutinosior CCTCC NO: M 2019414 of Example 1.

FIG. 2 is a photograph of Basidiospores under electron microscope of Pholiota glutinosior CCTCC NO: M 2019414 of Example 1.

FIG. 3 is a photograph of Basidia and Basidiospores under general optical microscope of Pholiota glutinosior CCTCC NO: M 2019414 of Example 1.

FIG. 4 is a phylogenetic tree constructed by the BI method based on ITS of Example 1.

FIG. 5 is a phylogenetic tree constructed by the ML method based on ITS of Example 1.

FIG. 6 is a phylogenetic tree constructed by the NJ method based on ITS of Example 1.

FIG. 7 is a phylogenetic tree constructed by the BI method based on the LSU of Example 1.

FIG. 8 is a phylogenetic tree constructed by the BI method based on RPB2 of Example 1.

FIG. 9 is a fruit body diagram of artificially domesticated new species of genus Pholiota glutinosior of Example 2.

FIG. 10 is another diagram of artificially domesticated new species of genus Pholiota glutinosior of Example 2.

FIG. 11 is inhibitory zone of the strain W140054 of the present invention of Example 3.

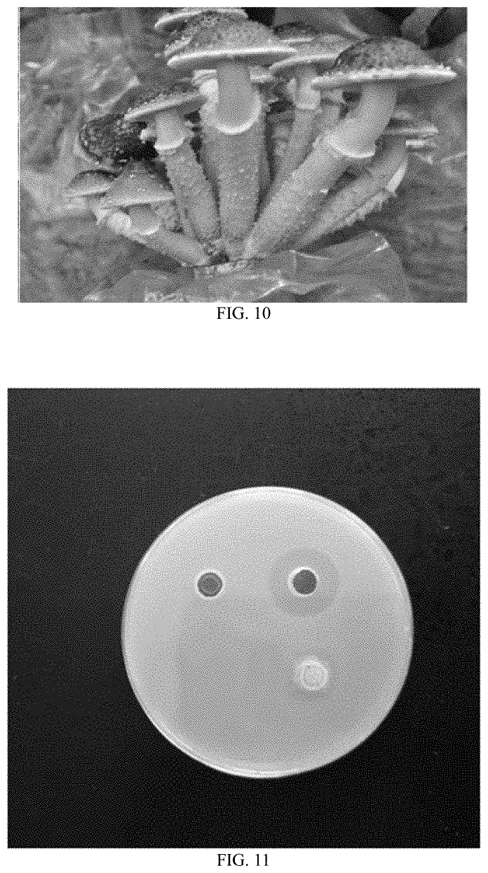

In FIG. 11, the upper left is a negative control; the upper right is a positive control; the lower right is the strain W140054 of the present invention.

### DETAILED DESCRIPTION

The Following is further illustration with the specific examples.

In the first aspect, the new strain of genus Pholiota of the present invention is collected from the Guangdong Chebaling National Nature Reserve, and is identified as a new strain of genus Pholiota, and the original strain is obtained by tissue separation, and is named as HMGIM-W140054, deposited on Jun. 3, 2019, at China Center for Type Culture Collection (CCTCC, Wuhan, China), with the accession number CCTCC NO: M 2019414.

The ITS sequence of its fruiting body has the highest similarity with Pholiota limonella (Registration Number: KM496470, From Korea), which is 99.2-99.7%, and has the 98.5-98.7% similarity with Pholiota adiposa (Registration Number: FJ810180, From China). Combined with the morphological identification, the macroscopic morphology of the strain is not consistent with Pholiota limonella and Pholiota adiposa. The difference is large. The shape of Pholiota adiposa is larger than that of the present invention more than 2 times, the Basidiospores of Pholiota limonella have obvious bud holes, but the species of the invention do not have bud holes, and Basidiospores of this species are smaller than Pholiota limonella. Therefore, it was judged that the strain of the present invention is a new species different from the current species of genus Pholiota, and was named as Pholiota glutinosior.

The macroscopic morphology and microscopic morphology of Pholiota glutinosior of the present invention are as follows: Basidiomata small sized. Pileus (0.65)1.0-2.2 cm in diameter, convex to bell shape when young and the edge of the pileus becoming flat, Orange-yellow, the middle color is darker, no obvious mucus, but it has strong viscous. The middle part of the cap has yellow-brown small spots, and the edge of the cap is wound inside, leaving white fluffy flakes. Stipe. central, 1.1˜4.8 cm, Stick-shaped, fleshy, pale yellow to white, nearly equal in thickness, slightly enlarged and curved at the base, with villiform annulus; the stipe above the annulus is smooth and nearly white, and the stipe below the annulus is light yellow and covered with fluff flakes. Hymenophore. lamella white to off-white color, adnate, sparse, unequal length with thickness is about 0.8˜1.0 mm. Basidiospores. 5.57˜7.11×3.46˜4.38 μm, Q=1.68, long-oval, smooth, with yellowish brown color, partially with oily, bud germ pores are not obvious, non-amyloid. Basidia. 17.8˜25.24×6.8˜7.52 μm, clavate without basal clamp connection, sterigmata up to 23 μm. Cheilocystidia. 16.35˜28.24×7.15˜10.0 μm, long stick shape, smooth without attachments. Pleurocystidia. 18.71˜26.1×6.47˜8.62 μm, short rod to spindle with a short-pointed. Clamp connections were present in tissue of context.

In the second aspect, the present invention provides an artificial cultivation method of Pholiota glutinosior CCTCC NO: M 2019414, which comprises producing the first-class strain, producing the second-class strain, producing the third-class strain, cultivating culture and cultivation management, by weight percent, the cultivating medium comprises 28-32% cottonseed hulls, 56-58% wood chips, 8-12% bran, 1-2% CaCO₃.

Preferably, the above cultivating medium has moisture content of 60%-65%.

Preferably, by weight percent, the cultivating medium comprises 31% cottonseed hulls, 58% wood chips, 10% bran, 1% CaCO₃, and has moisture content of 60% to 65%.

Preferably, the cultivation comprises: transferring the third-class strain to the cultivating medium, keeping constant temperature at 25-26° C., shading culture, humidity 60%-70%, and the hyphae overgrowing the cultivation bag means it is entering into cultivation management.

Preferably, the cultivation management comprises:

after the hyphae in the bag is full of the cultivating medium in the bag, the cultivation continues for 25 days after the shading, and entering the mushroom stage, the temperature is controlled at 18-20° C., and the ventilation is increased to keep the space carbon dioxide content below 1%. The relative humidity of the air is adjusted to more than 90%. After 14 days, the cap is removed and the hyphae begins to kink and forms the pale yellow rice bran-like primordium. After the primordium grows to 0.5 cm, the temperature is kept at 18-20° C., relative humidity between 80-90%, 9 hours of light per day, light intensity 300-500 lx, and keep the carbon dioxide concentration in the air 350˜1500 ppm, keep the air moist, after about 8 days, during this period, spraying the mushroom every day 1-2 times until the size of the fruit body is basically unchanged, and the fruit body cap no longer grows and begins to flatten, then harvest.

Preferably, the preparation of the first-class strain comprises: transferring the isolated strains to the first-class strain culture medium, and placing them in a constant temperature dark culture at 25° C. When the mycelium grows and the bacteria have not grown, the tip hyphae is picked up to obtain the first-class strain.

Preferably, the first-class strain culture medium is Bengal red medium.

Further preferably, by weight percent, the Bengal red medium comprises: peptone 0.5%, glucose 1%, potassium dihydrogen phosphate 0.1%, magnesium sulfate (MgSO₄.7H₂O) 0.05%, agar 2%, 1/3000 Bangladesh Red solution 10%, and chloramphenicol 0.01%, the rest is water.

Preferably, the production of the second-class strain comprises: transferring the first-class strain to the second-class strain culture medium, and placing it in a constant temperature dark culture at 25° C., and the mycelium is full of slopes as to obtain the second-class strain

Preferably, the second-class strain is an enriched integrated potato dextrose agar.

Further preferably, by weight percent, the enriched integrated potato dextrose agar comprises: 20% potato, 20% glucose, 1% peptone, 2% agar, 0.3% potassium dihydrogen phosphate, 0.15% magnesium sulfate, and 0.15% vitamin B1, and the rest is water.

Preferably, the production of the third-class strain comprises: inoculating the second-class strain into the third-class strain culture medium, ensuring that the second-class strain material block is buried in the first-class strain culture medium, and is placed in a constant temperature dark culture at 25° C. until the hyphae full of medium, the third-class strain is obtained.

Preferably, by weight percent, the third-class strain culture medium comprises: 98-99% sorghum and 1-2% calcium carbonate.

Further preferably, by weight percent, the third-class strain culture medium comprises: 98% sorghum and 2% calcium carbonate.

Preferably, the artificial cultivation method further comprises tissue strain isolation before the preparation of the first-class strain.

An artificial cultivation method of Pholiota glutinosior CCTCC NO: M 2019414, which comprises producing the first-class strain after tissue isolation, producing the first-class strain, producing the second-class strain, producing the third-class strain, cultivating culture and cultivation management, by weight percent, the cultivating medium comprises 28-32% cottonseed hulls, 56-58% wood chips, 8-12% bran, and 1-2% CaCO₃.

Preferably, the tissue-separating strain comprises: wiping the surface of fruit body with alcohol under aseptic conditions, tearing, and vaccinating the internal tissue of 0.2-0.5 mm×0.2-0.5 mm aseptically, placing in a constant temperature dark culture at 25° C., and the isolated strain was obtained after the slant were covered with hyphae.

Preferably, the tissue isolation medium is the integrated potato dextrose agar medium.

Further preferably, by weight percent, the integrated potato dextrose agar medium comprises 20% potato, 2% glucose, 2% agar, 0.3% potassium dihydrogen phosphate, 0.15% magnesium sulfate, and a trace amount of vitamin B1.

In the third aspect, the present invention provides the use of a new species Pholiota glutinosior CCTCC NO: M 2019414 or an extract thereof for use in diseases associated with anti-bacteria.

Preferably, the extract is the ethyl acetate extract.

Preferably, the bacterium is Staphylococcus, further preferably Staphylococcus aureus.

In the fourth aspect, the present invention provides the use of a new specie Pholiota glutinosior CCTCC NO: M 2019414 or an extract thereof for preparation of a medicament for treating diseases caused by anti-bacteria or for preparation of the health supplement.

Preferably, the extract is the ethyl acetate extract.

Preferably, the bacterium is Staphylococcus, further preferably Staphylococcus aureus.

Preferably, for the preparation of the health supplement comprises for the preparation of health supplement rich in histidine, and/or crude polysaccharide, and/or potassium content.

In the fifth aspect, the present invention provides the medicament for treating diseases caused by anti-bacteria, comprising a new specie Pholiota glutinosior CCTCC NO: M 2019414 or an extract thereof and a carrier.

Preferably, the extract is the ethyl acetate extract.

Preferably, the bacterium is Staphylococcus, further preferably Staphylococcus aureus.

In the sixth aspect, the present invention provides the health supplement comprising a new specie Pholiota glutinosior CCTCC NO: M 2019414 or an extract thereof.

Preferably, the health supplement is the health supplement rich in histidine, and/or crude polysaccharide, and/or potassium.

### Example 1: Identification of New Strain

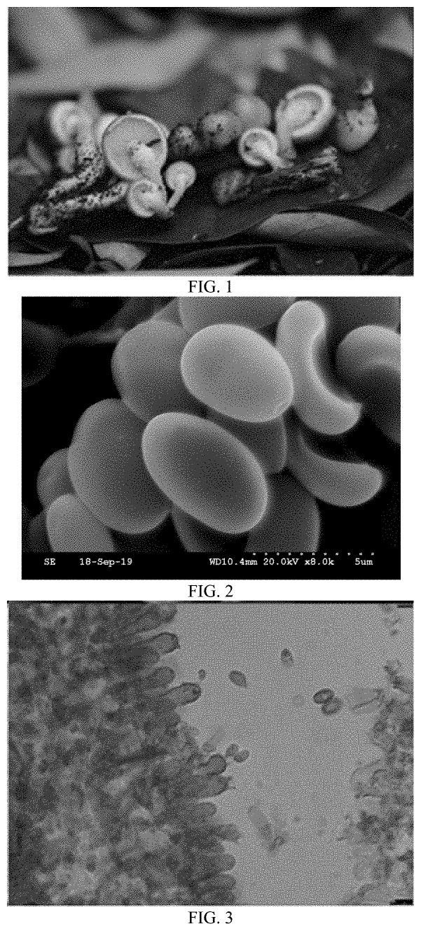

On Mar. 22, 2014, Hu Huiping and Liu Yuanchao collected and investigated large-scale fungal resources in the Chebaling National Nature Reserve, Guangdong Province. A specimen of genus Pholiota was collected on the dead wood, as shown in FIG. 1. FIG. 2 and FIG. 3 are micrographs of the structure, and the original strain was obtained by tissue isolation, named HMGIM-W140054, deposited on Jun. 3, 2019, at China Center for Type Culture Collection (CCTCC, Wuhan, China), with the accession number CCTCC NO: M 2019414.

Since it did not find the species of genus Pholiota that was consistent with its description in the macroscopic and microscopic morphology, in order to further confirm its classification, DNA extraction was performed, and the genes of the multiple fragments were sequenced, including the fungal ribosomal intergenic region ITS, ribosomal large subunit LSU, a second large subunit RPB2 encoding RNA polymerase II, and the like, which are used for fungal classification of polygenic fragments.

The researchers in the Edible Fungus Research and Development Center of the Guangdong Institute of Microbiology conducted a low-temperature (35° C.) drying of wild fruiting bodies, grinded by liquid nitrogen, extracted the DNA genome using the Ezup column fungal genomic DNA extraction kit. The DNA solution was chilled at −20° C. for use. Universal primer for fungal ribosomal intergenic region (ITS) ITS1/ITS4 (ITS1: TCC GTA GGT GAA CCT GCG G, ITS4: TCC TCC GCT TAT TGA TAT GC, ribosomal large subunit (LSU) primer LROR/LR7 (LROR): ACC CGC TGA ACT TAA GC, LR7: TAC TAC CAC CAA GAT CT), the second large subunit (RPB2) primer encoding RNA polymerase II RPB2-B-F1/RBP2-BR (RPB2-B-F1: AAG ATY GCY AAG CCT CGT CA, RBP2-BR: AAG ATR TTG GCC ATS GTG TCC) (synthesized by Shanghai Meiji Biomedical Technology Co., Ltd.) PCR experiments on materials, amplification on Biometra PCR instrument, PCR reaction composition (50μl total) is:

TaKaRaTaq (5 units/μl) 0.25 μl

10×PCR Buffer 5 μl

dNTP Mixture (2.5 mM each) 4 μl

DNA template 2 μl

Primer 1 (10 μmol·L−1) 5 μl

Primer 2 (10 μmol·L−1) 5 μl

Sterilized distilled water 28.75 μl

The ITS-PCR reaction condition is: reaction at 94° C. for 5 min; reaction at 94° C. for 1 min, reaction at 55° C. for 1 min, reaction at 72° C. for 1 min, 30 cycles; reaction at 72° C. for 10 min. The PCR product was directly sent for bidirectional sequencing and was completed by Shanghai Meiji Biomedical Technology Co.

Due to the lack of bases in ITS-PCR, Shanghai Meiji Biomedical Technology Co., Ltd. was further commissioned to carry out cloning and sequencing, and six clones and their sequencing results were obtained. Three sequences were uploaded to Genebank, and the registration serial numbers were MN582988 (SEQ ID NO: 1), MN582989 (SEQ ID NO: 2), and MN582990 (SEQ ID NO: 3).

The reaction condition of LSU-PCR is: reaction at 95° C. for 3 min; reaction at 94° C. for 30 s, reaction at 50° C. for 45 s, reaction at 72° C. for 2 min, 36 cycles; reaction at 72° C. for 10 min. The obtained LSU sequence was uploaded to Genebank with the registration serial number MN582991 (SEQ ID NO: 4).

The reaction condition of RPB2-PCR is: reaction at 95° C. for 3 min; reaction at 95° C. for 30 s, reaction at 55° C. for 45 s, reaction at 72° C. for 1.5 min, 36 cycles; reaction at 72° C. for 10 min. The three RPB2 sequences obtained were uploaded to Genebank with accession numbers MN628554 (SEQ ID NO: 5), MN628555 (SEQ ID NO: 6), MN628556 (SEQ ID NO: 7).

The newly-generated sequences were checked and then submitted to GenBank. Phylogenetic trees were built separately based on ITS, LSU and RPB2, Kuehneromyces mutabilis and Hypholoma fasciculare were selected as outgroup respectively. All sequences were aligned with CLUSTAL W (v.1.83) using default settings. Phylogenetic analysis of ITS, LSU and RBP2 were performed using Bayesian algorithm (BI), the parameters was set as follows: the analysis was run in MrBayes 3.1.2 (Ronquist & Huelsenbeck, 2003), mcmcp ngen=1000000, sample freq.=100. The NJ and ML Phylogenetic trees of ITS were also performed using MEGA (V10.0.4).

Phylogenetic trees of ITS: Kuehneromyces mutabilis data (AY354218) were choosed as outgroup, phylogenetic trees was constructed on ITS data. The ITS data included sequences from fifteen Pholiota samples representing seven taxa. The best model selected and applied in the Mybayes analysis for the ITS model. BI analysis resulted with an average standard deviation of split frequencies=0.006934. Sequences of the fruit body of HMGIM W140054 had been deposited in GenBank and accession numbers is MN582988, MN582989 and MN582990.

Phylogenetic trees of LSU: Kuehneromyces mutabilis data (MH866740) were choosed as outgroup, phylogenetic trees was constructed on LSU data. The LSU data included sequences from twelve Pholiota samples representing seven taxa. The best model selected and applied in the Mybayes analysis for the ITS model. BI analysis resulted with an average standard deviation of split frequencies=0.003046. Sequences of the fruit body of HMGIM W140054 had been deposited in GenBank and accession numbers is MN582991.

Phylogenetic trees of RPB2: Hypholoma fasciculare data (AY337413) were choosed as outgroup, phylogenetic trees was constructed on RPB2 data. The RPB2 data included sequences from seven Pholiota samples representing four taxa. The best model selected and applied in the Mybayes analysis for the ITS model. BI analysis resulted with an average standard deviation of split frequencies=0.002288. Sequences of the fruit body of HMGIM W140054 had been deposited in GenBank and accession numbers is MN628554, MN628555, MN628556.

Species, specimens, geographic origin and GenBank accession numbers of sequences used in this study are shown in Table 1.

TABLE 1
Species, specimens, geographic origin and GenBank accession numbers of sequences used in this study

| | | | GenBank accession numbers | | | |
|---|---|---|---|---|---|---|
| Species name | Voucher no. | Country | ITS | LSU | RPB2 | References |
| Pholiota adiposa | CBS 279.29 | Netherlands | MH855073 | MH866533 | | Vu, D, 2019 (Vu et |
| | | | | | | al., 2019) |
| P. adiposa | CBS 561.87 | Netherlands | MH862102 | MH873791 | | Vu, D, 2019 |
| P. baeosperma | TFB8315 | USA | | | MF978336 | Matheny, P. B, |
| | | | | | | 2018 (Matheny, |
| | | | | | | Swenie, Miller, |
| | | | | | | Petersen, & |
| | | | | | | Hughes, 2018) |
| P. baeosperma | TFB7383 | USA | | | MF978357 | Matheny, P. B, |
| | | | | | | 2018 |
| P highlandensis | PBM4085 | USA | | | MH360729 | Matheny, P. B, |
| | | | | | | 2018 |
| P. highlandensis | FIRE409 | USA | | | MH360728 | Matheny, P. B, |
| | | | | | | 2018 |
| P. lenta | MCVE 7100 | Italy | JF908582 | | | Osmundson, |
| | | | | | | T. W., 2013 |
| | | | | | | (Osmundson et al., |
| | | | | | | 2013) |
| P. lenta | CBS 182.53 | Netherlands | MH857154 | MH868690 | | Vu, D, 2019 |
| P. lenta | GLM 45997 | Germany | | AY207270 | | Walther, G., 2005 |
| | | | | | | (Walther, Garnica, |
| | | | | | | & Weiss, 2005) |
| P. lenta | CBS 185.53 | Netherlands | MH857156 | MH868692 | | Vu, D, 2019 |
| P. limonella | G0779 | Hungary | | MK278459 | | Varga, T, 2019 |
| | | | | | | (Varga et al., |
| | | | | | | 2019) |
| P. limonella | KUC20130923-06 | Korea | KM496470 | | | Jang, S, 2014, |
| | | | | | | Unpublished |
| P. limonella | SFC20150707-19 | South Korea | KX773882 | | | Cho, H. J, 2016 |
| | | | | | | (Cho et al., 2016) |
| P. lubrica | PRM 857179 | Slovakia | HG007984 | | | Holec, J, 2014 |
| | | | | | | (Holec et al., |
| | | | | | | 2014) |
| P. lubrica | | China | JF961353 | | | Tian, E. J, 2011, |
| | | | | | | Unpublished |
| P. lubrica | G0252 | Hungary | | MK278460 | | Varga, T, 2019 |
| P. microspora | CBS 360.51 | Netherlands | MH856901 | MH868423 | | Vu, D, 2019 |
| P. microspora | ZJ0005QGG01 | China | KU836562 | | | Liu, Y, 2016, |
| | | | | | | Unpublished |
| P. multicingulata | PDD97861 | New Zealand | | | HQ832440 | Matheny, P. B., 2010, |
| | | | | | | Unpublished |
| P. glutinosior | HMGIM-W140054-1 | China | MN582988* | | | This study |
| P. glutinosior | HMGIM-W140054-2 | China | MN582989* | | | This study |
| P. glutinosior | HMGIM-W140054-3 | China | MN582990* | | | This study |
| P. glutinosior | HMGIM-W140054 | China | | MN582991* | | This study |
| P. glutinosior | HMGIM-W140054-4 | China | | | MN628554* | This study |
| P. glutinosior | HMGIM-W140054-5 | China | | | MN628555* | This study |
| P. glutinosior | HMGIM-W140054-6 | China | | | MN628556* | This study |
| P. spumosa | MCVE3533 | Italy | JF908577 | | | Osmundson, |
| | | | | | | T. W, 2013 |
| P. spumosa | CBS 245.50 | France | MH856605 | | | Vu, D, 2019 |
| P. spumosa | G0436 | Hungary | | MK278465 | | Varga, T, 2019 |
| P. spumosa | | Germany | | AY207272 | | Walther, G, 2005 |
| | | | | | | (Walther et al., |
| | | | | | | 2005) |
| P. spumosa | PBM2849 | USA | | | MG923698 | Matheny, |
| | | | | | | P. B, 2018 |
| P. spumosa | ZRL20151729 | China | | | KY419028 | Zhao, R. -L, 2017 |
| | | | | | | (Zhao et al., 2017) |
| P. squarrosoides | MCVE17140 | Finland | JF908591 | | | Osmundson, |
| | | | | | | T. W., 2013 |
| P. squarrosoides | BHI-F081b | USA | MF161170 | | | Haelewaters, D, |
| | | | | | | 2018 |
| | | | | | | (Haelewaters, |
| | | | | | | 2018) |
| P. squarrosoides | SV.S1 | USA | | AF261641 | | Moncalvo, |
| | | | | | | J. -M, 2002 |
| | | | | | | (Moncalvo et al., |
| | | | | | | 2002) |
| P. squarrosoides | G0510 | Hungary | | MK278467 | | Varga, T, 2019 |
| Kuehneromyces | olrim351 | Lithuania | AY354218 | | | Lygis, V, 2004 |
| mutabilis | | | | | | (Lygis, |
| | | | | | | Vasiliauskas, & |
| | | | | | | Stenlid, 2004) |
| Kuehneromyces | CBS 205.32 | Netherlands | | MH866740 | | Vu, D, 2019 |
| mutabilis | | | | | | |
| Hypholoma | PBM 1844 | USA | | | AY337413 | Matheny, |
| fasciculare | | | | | | P. B, 2005 |

*Newly generated sequences for this invention.

The clone sequencing results of ITS conducted sequenced Blast in GenBank. The six clones were the most similar to Pholiota limonella (registration number: KM496470, From Korea), which is 99.2-99.7%, and has the 98.5-98.7% similarity with Pholiota adiposa (Registration Number: FJ810180, From China). The macroscopic morphology of the strain is not consistent with Pholiota limonella and Pholiota adiposa. The difference is large. The shape of Pholiota adiposa is larger than that of the present invention more than two times, Basidiospores of Pholiota limonella have obvious bud holes, but the species of the invention don't have bud holes, and Basidiospores of this species are smaller than Pholiota limonella. Therefore, it was judged that the strain of the present invention is a new species different from the current species of genus Pholiota.

The highest similarity of the LSU alignment results was Pholiota squarrosa, which is 98.89% (Registration number: DQ470818, From the United States), apparently not the same strain.

The RPB2 alignment results have the highest similarity between 86-88% substantially, and are all species of genus Pholiota, which are different from the varieties in the present invention.

Observing its macroscopic morphology and microscopic morphology: Basidiomata small sized. Pileus (0.65)1.0-2.2 cm in diameter, convex to bell shape when young and the edge of the pileus becoming flat, Orange-yellow, the middle color is darker, no obvious mucus, but it has strong viscous. The middle part of the cap has yellow-brown small spots, and the edge of the cap is wound inside, leaving white fluffy flakes. Stipe. central, 1.14.8 cm, Stick-shaped, fleshy, pale yellow to white, nearly equal in thickness, slightly enlarged and curved at the base, with villiform annulus; the stipe above the annulus is smooth and nearly white, and the stipe below the annulus is light yellow and covered with fluff flakes. Hymenophore. lamella white to off-white color, adnate, sparse, unequal length with thickness is about 0.81.0 mm. Basidiospores. 5.57˜7.11×3.46˜4.38 μm, Q=1.68, long-oval, smooth, with yellowish brown color, partially with oily, bud germ pores are not obvious, non-amyloid. Basidia. 17.8˜25.24×6.8˜7.52 μm, clavate without basal clamp connection, sterigmata up to 23 μm. Cheilocystidia. 16.35˜28.24×7.15˜10.0 μm, long stick shape, smooth without attachments. Pleurocystidia. 18.71˜26.1×6.47˜8.62 μm, short rod to spindle with a short-pointed. Clamp connections were present in tissue of context.

According to its morphology, the species that is currently consistent with that is not found. The closest one is Pholiota lubrica, but these two are obviously inconsistent in color, Pholiota lubrica is reddish-brown, and the color of the middle and the edge is different. The color is bright yellow and uniform. Moreover, the individuals of Pholiota lubrica are slightly larger, usually distributed in the northeast and the middle of China, and they are far apart from the ITS sequences of these two. Basically, this species is not Pholiota lubrica.

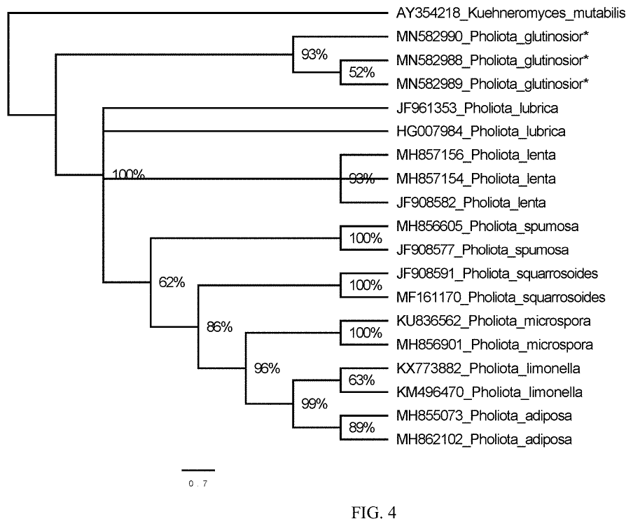

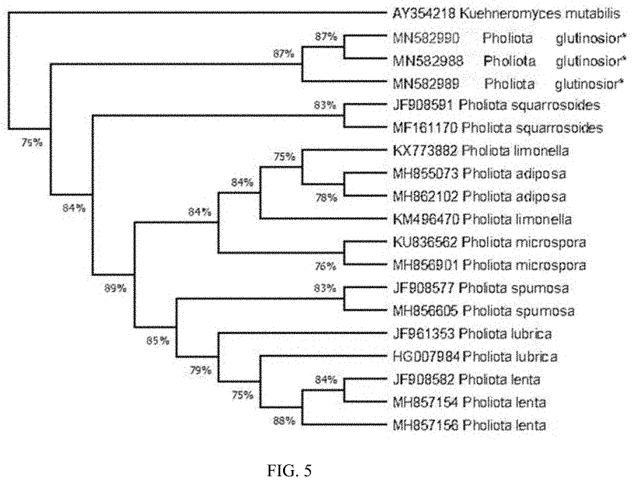

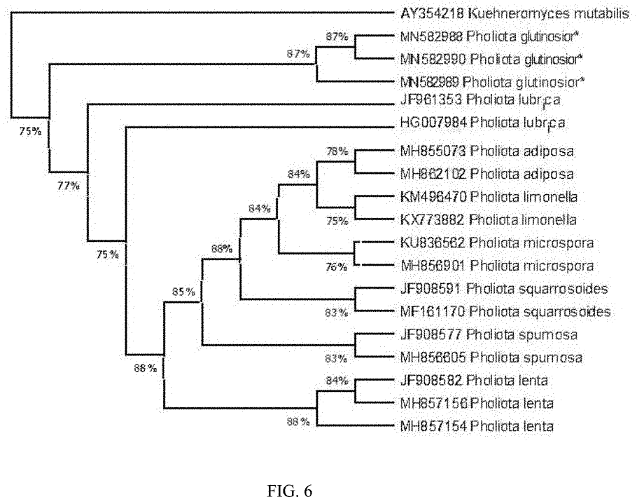

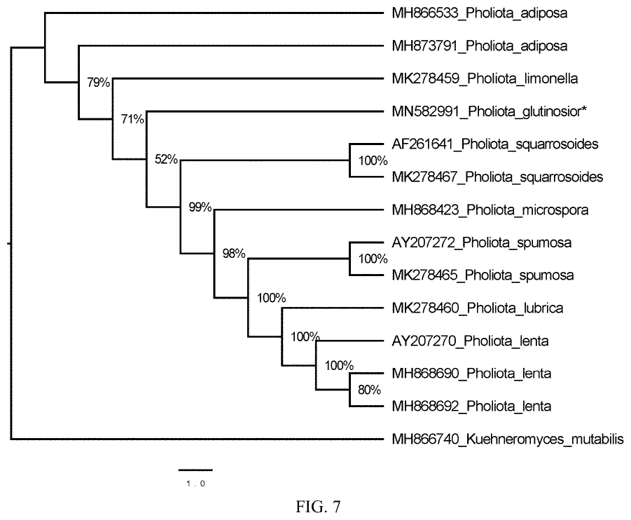

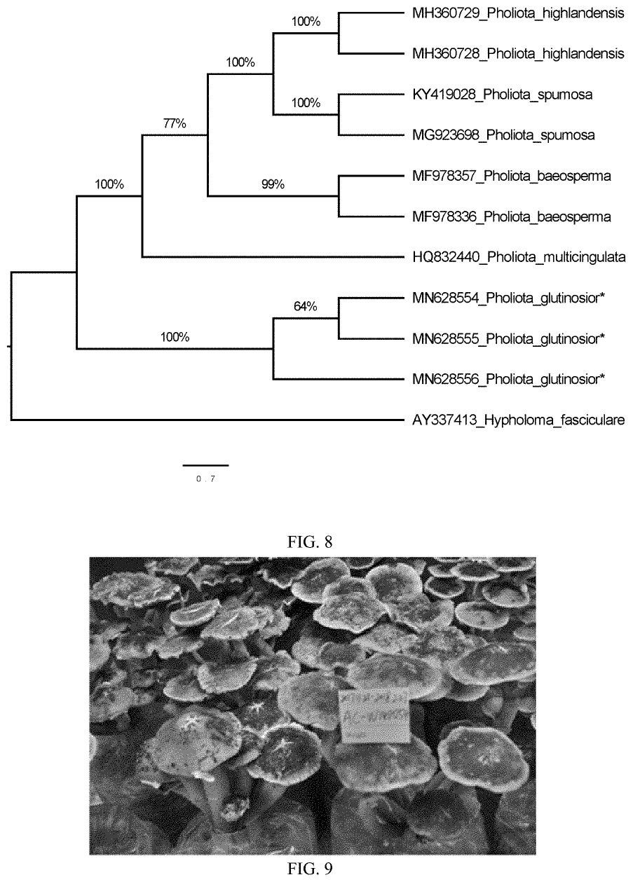

In order to further confirm its species, we searched the literature for the credible IBS sequence, LSU sequence and RPB2 sequence, using Bayesian method (BI), neighboring method (NJ) and maximum likelihood method (ML), constructed the phylogenetic tree using the Kuehneromyces mutabilis and the clustered Hypholoma fasciculare as the outer group, respectively, as shown in FIG. 4-8.

From the results of these three phylogenetic trees in FIG. 4 to FIG. 8, the results are consistent. The sequencing results of genus Pholiota are clustered together, indicating that the sequencing results are credible. The highest alignment similarity is Pholiota limonella and Pholiota adiposa for ITS. For LSU, the highest alignment similarity is Pholiota squarrosa. The highest macroscopic and microscopic similarity is Pholiota lubrica. However, in the phylogenetic trees, it cannot be clustered with these species, and the genetic distance is very far. At the same time, its macroscopic and microscopic morphology indicates that it is a species of genus Pholiota, so we judge it as a new species, named Pholiota glutinosior.

### Example 2 Artificial Cultivation

1. Medium (by Weight Percent):

(1) Tissue Separation Medium (Integrated Potato Dextrose Agar):

Potato 20%, glucose 2%, agar 2%, potassium dihydrogen phosphate 0.3%, magnesium sulfate 0.15%, and vitamin B1 trace, the rest is water.

(2) Purified First-Class Strain Medium (Bengal Red Medium):

Peptone 0.5%, glucose 1%, potassium dihydrogen phosphate 0.1%, magnesium sulfate (MgSO₄.7H₂O) 0.05%, agar 2%, 1/3000 Bengal red solution 10%, and chloramphenicol 0.01%, and the rest was water.

(3) Second-Class Strain Culture Medium (Plus Rich Potato Dextrose Agar):

Potato 20%, glucose 2%, peptone 1%, agar 2%, potassium dihydrogen phosphate 0.3%, magnesium sulfate 0.15%, and vitamin B1 trace, the rest is water.

(4) Third-Class Strain Culture Medium:

98-99% sorghum, and 1-2% calcium carbonate.

(5) Cultivation Materials:

31% cottonseed hulls, 58% wood chips, 10% bran, and 1% CaCO₃; moisture content 60%-65%.

2. Method:

(1) Tissue Isolation Strains:

The tissue separation medium was prepared, and the medium was dispensed into tube, and sterilized by heat sterilization at 0.11 MPa atmospheric pressure and 121° C. high temperature and high pressure for 30 min, and taken out to cool and be inclined. The collected wild Pholiota fruit body was wiped with 75% alcohol under aseptic conditions, then teared, and 0.2-0.5 mm×0.2-0.5 mm of internal fungus tissue was inoculated into the medium aseptically. Place it in a 25° C. incubator at a constant temperature and dark culture. After the hyphae grows over the slope, it can be transferred. The period is between 10 days and 15 days.

(2) Production of the Purified First-Class Strain:

The purified medium was prepared according to the formula, and the medium was dispensed into tube, and sterilized by heat sterilization at 0.11 MPa atmospheric pressure and 121° C. for 30 minutes, and the fungus infected with the bacteria were transferred. It was placed in a 25° C. incubator at a constant temperature for dark culture. When the mycelium grew and the bacteria had not grown, the tip hyphae was picked and transferred to obtain the purified first-class strain.

(3) Production of the Second-Class Strain:

The second-class strain culture medium was prepared, and the medium was dispensed into tube, and sterilized by heat sterilization at 0.11 MPa atmospheric pressure and 121° C. for 30 minutes, and cooled down, and then incubated the purified first-class strain which was successfully separated under aseptic operation. Place it in a 25° C. incubator at a constant temperature and dark culture. After the hyphae grows over the slope, the second-class strain can be obtained and then transferred. The period for the second-class strain overgrowing is about 15 days to 20 days.

(4) Production of the Third-Class Strain

Weigh the required proportion of sorghum, wet it overnight with water, mix it with calcium carbonate in proportion, put it into 250 ml Erlenmeyer flask with 100-150 g of dry material per bottle, get the third-class strain culture medium, seal with silicone plug, sterilize at 0.147 MPa atmospheric pressure, 128° C. with high temperature, high pressure and humid heat for 90 min, take out and cool down, the medium was shaken and then be inoculated with the second-class strain aseptically. At the time of inoculation, it is ensured that the second-class strain block is buried in the third-class strain stock. Place it in a 25° C. incubator at a constant temperature and dark culture. After the hyphae is full (twenty days or so), it can be used as the third-class strain inoculated in the cultivation bag.

(5) Cultivation

The required proportion of culture material by the artificial domestication medium is taken, thoroughly mixed and added with water (water content of 55-65%), and filled into a 17 cm×35 cm high transparent polypropylene cultivation bag with 400-420 g per bag of dry material. After loading the material, make a hole in the bag material using a small wooden stick, and the hole is deep to the bottom of the bag. Then, a plastic ring is placed on the bag mouth, and the matching cover is buckled to obtain a prepared cultivation bag. The mixture was sterilized by heat sterilization at 0.147 MPa atmospheric pressure and 128° C. for 90 minutes. Ensure that the block is buried in the cultivation material during inoculation. After inoculation, the cultivation bags were cultured in the dark at 25° C.±1° C. and air relative humidity of 60-70%. After the hyphae are full (about 25 days), it can enter the cultivation management.

(6) Cultivation Management (Including after-Ripening Management, Primordium Formation, Fruiting Body Growth)

\textcircled{1} after-Ripening Management

After the hyphae in the bag is full of the cultivation material in the bag, the shading is continued for 25 days and then matured for 25 days to enter into the mushrooming stage.

\textcircled{2} Primordium Formation

Control the temperature at 18-20° C., and increase the ventilation, keep the space carbon dioxide content below 1%, the air relative humidity is adjusted to more than 90%, after about 14 days, remove the cap and put the cultivation bag vertically (gap between the bags). After about 14 days, the hyphae began to kink and form the pale yellow rice bran-like primordium.

\textcircled{3} Fruiting Body Growth Period

After the primordium grows to 0.5 cm, continue to control the temperature at 18-20° C., air relative humidity 80-90%, 9 hours of light per day, light intensity 300-500 lx, and maintain the carbon dioxide concentration in the air 350˜1500 ppm, keep the air moist. It can be harvested after about 8 days. During this period, the water is sprayed 1-2 times a day to the young mushroom until the size of the fruit body is basically unchanged. The fruit body cap no longer grows and begins to flatten, indicating that the fruiting body has matured and should be harvested at this time.

It takes about 8 days from the growth of the primordial to the maturity of the fruiting body. After harvesting the mushroom firstly, the same mushroom treatment was carried out, and the mushroom was harvested secondly after about 25 days. The results are shown in FIG. 9 and FIG. 10.

3. Situation of Mushrooms

(1) Fruiting period: The first and second mushroom periods total are 90 days, and the first mushroom period is 65 days.

(2) Yield: The average bag yield is 139.71 grams.

(3) Fruiting body traits: Basidiomata small sized. Pileus (0.65)1.0-2.2 cm in diameter, convex to bell shape when young and the edge of the pileus becoming flat, Orange-yellow, the middle color is darker, no obvious mucus, but it has strong viscous. The middle part of the cap has yellow-brown small spots, and the edge of the cap is wound inside, leaving white fluffy flakes. Stipe. central, 1.1˜4.8 cm, Stick-shaped, fleshy, pale yellow to white, nearly equal in thickness, slightly enlarged and curved at the base, with villiform annulus; the stipe above the annulus is smooth and nearly white, and the stipe below the annulus is light yellow and covered with fluff flakes. Hymenophore. lamella white to off-white color, adnate, sparse, unequal length with thickness is about 0.8˜1.0 mm.

Compared with the wild state, the individual fruit body of the species increased significantly after artificial domestication, and the fruiting body was relatively neat and the yield was high.

After artificial domestication, it can be seen that the traces of charcoal burning adhered to the caps of the mature fruiting bodies, which are significantly different from the species Pholiota glutinosior already described, and it is also confirmed as a new species from the side.

### Example 3 Determination of Antibacterial Activity of Fermentation Broth

1. Method

(1) Preparation:

Bacterial medium: nutrient agar/broth medium;

Nutrition agar/broth medium: beef paste 3 g, peptone 10 g, sodium chloride 5 g, agar 15 g, add water to 1000 ml, and adjust the pH to 7.4 (nutrient broth without agar).

Preparation of ethyl acetate extract: using rice medium (formulated rice and water), the strain of the present invention was incubated and cultured for 45 days, ethyl acetate was added, ultrasonic extraction was performed twice, 40 minutes for each time, and the extract was combined. The solvent was recovered in a rotary evaporator, and steamed to about 1-2 mL of the extract, and transferred to a collection bottle, the ethyl acetate extract is obtained and placed in a vacuum desiccator. After the solvent was completely evaporated, it was transferred to a refrigerator at 4° C. for use, and was filtrated and sterilizd with the bacteria syringe and filter for use.

Positive control preparation: Ampicillin solution, diluted with sterile water to a concentration of 5 μg/mL of the control solution, UV sterilization for 30 min.

(2) Preparation of Bacteria Suspension

\textcircled{1} The Staphylococcus aureus cryopreservation solution was taken out from the −80° C. refrigerator, and a single colony was obtained by streaking the plate.

\textcircled{2}A single colony was picked up with a sterile inoculating loop and inoculated in a nutrient broth medium, and cultured at 37° C., 220 r/min for 24 h, to obtain a bacteria suspension for the test bacteria.

\textcircled{3} Sucking the bacteria suspension, and diluting with sterile distilled water to obtain the bacteria liquid having a concentration of 10⁻¹ to 10⁻⁸.

\textcircled{4} 200 μL of each concentration of the bacteria solution was uniformly applied to the nutrient agar plate, cultured at 37° C. for 24 h, and each concentration was repeated 3 times.

\textcircled{5} Determine the concentration of the bacteria solution by the plate counting method, and waiting for use.

(3) Determination of Inhibition Zone

\textcircled{1} Coating Method

The counted bacteria solution was diluted to a final concentration of 10⁵˜10⁶ cfu/mL, and 200 μL of the prepared bacteria suspension was pipetted onto the plate and uniformly coated with a coater. Use a sterilized puncher to evenly punch holes in the bacteria-containing plate, carefully pick out the medium in the well, and pipett the control solution and 2004, of the test sample into the well under aseptic conditions, and place at 37° C. The culture was allowed to stand for 1 day, and the size of inhibition zone was measured.

\textcircled{2} Pre-Added Bacteria Solution Pouring Plate Method

Inject a certain amount of bacteria solution into the plate medium that has been cooled to about 50° C., so that the concentration of the bacteria in the medium is 10⁵-10⁶ cfu/mL, shake well, pour the plate (about 30 mL/plate), and let it stand horizontally to be coagulated. The method of punching and loading is the same as above.

(4) Data Processing

Measure and compare the size of inhibition zone.

2. Experimental Results:

The results are shown in FIG. 11 and Table 2.

TABLE 2
Diaphragm diameter of Staphylococcus aureus (x ± s, n = 3)

| Sample | inhibition zone diameter (mm) |
|---|---|
| Positive control | 19.79 ± 1.7 |
| The strain W140054 of the invention | 14.17 ± 0.79 |

As shown in FIG. 11, the fermentation broth of the strain exhibits an inhibition zone, indicating that the fermentation broth of the present invention has antibacterial activity against Staphylococcus aureus, and about 600 wild edible medicinal bacteria in the same batch are screened. Among the ethyl ester extracts, the strain is one of the top 20 strains with better effects, which indicates that the strain of the present invention has a certain inhibitory effect on Staphylococcus aureus.

### Example 4 Determination of Nutrients

For the artificial cultivation of a new species of genus Pholiota, the nutrient composition was determined, including hydrolysis of amino acids, polysaccharides, proteins and important trace elements. Pholiota adiposa and Pholiota microspora were used as controls, and the results are shown in Table 3.

TABLE 3
Nutritional ingredients determination table

| | | Pholiota | Pholiota | Pholiota | |
|---|---|---|---|---|---|
| | unit | glutinosior | adiposa | microspora | Detection method |
| Aspartic acid | g/100 g | 1.01 | 1.299 | 1.360 | GB/T |
| *threonine | | 0.56 | 0.679 | 0.811 | 5009.124-2016 |
| Serine | | 0.51 | 0.672 | 0.810 | |
| Glutamate | | 1.77 | 3.032 | 3.383 | |
| Glycine | | 0.50 | 0.760 | 0.782 | |
| Alanine | | 0.65 | 0.908 | 0.894 | |
| *Proline | | 0.60 | 0.645 | 0.683 | |
| *methionine | | 0.11 | 1.541 | 1.217 | |
| *isoleucine | | 0.44 | 0.531 | 0.631 | |
| *Leucine | | 0.73 | 0.961 | 0.926 | |
| Tyrosine | | 0.25 | 0.284 | 0.277 | |
| *Phenylalanine | | 0.48 | 0.751 | 0.697 | |
| Histidine | | 1.32 | 0.294 | 0.318 | |
| *lysine | | 0.63 | 0.859 | 0.795 | |
| Arginine | | 0.51 | 0.802 | 0.663 | |
| Proline | | 0.51 | 0.556 | 0.497 | |
| Hydrolyzed | | 10.6 | 14.574 | 14.744 | |
| amino acid sum | | | | | |
| Cystine | | 0.26 | — | — | HPLC (After |
| | | | | | oxidative |
| | | | | | hydrolysis, |
| | | | | | the sample is |
| | | | | | determined |
| | | | | | according to the |
| | | | | | chromatographic |
| | | | | | conditions of GB |
| | | | | | 5009.124-2016.) |
| *Tryptophan | | 0.21 | — | — | The sample was |
| | | | | | subjected to |
| | | | | | alkaline |
| | | | | | hydrolysis |
| | | | | | treatment and |
| | | | | | determined by |
| | | | | | HPLC. |
| Sum of essential | | 3.76 | 5.967 | 5.760 | |
| amino acids | | | | | |
| Crude polysaccharide | g/100 g | 7.55 | — | 3.02 | NY/T |
| | | | | | 1676-2008/7 |
| protein | g/100 g | 14.9 | 17.02 | 18.10 | GB/T |
| | | | | | 5009.5-2016/ |
| | | | | | First method |
| calcium | mg/100 g | 4.01 | — | 42.1 (NY/ | GB/T |
| | | | | T1653- | 5009.268-2016 |
| | | | | 2008 Yao | Second method |
| | | | | Xingyu, 2017) | |
| Potassium | g/100 g | 2730 | — | 1340 (NY/ | GB/T |
| | | | | T1653- | 5009.268-2016 |
| | | | | 2008 Yao | Second method |
| | | | | Xingyu, 2017) | |
| magnesium | mg/100 g | 73 | 78.93 | 90.30 | GB/T |
| | | | | | 5009.268-2016 |
| | | | | | Second method |
| iron | mg/100 g | 3.16 | 50.77 | 53.88 | GB/T |
| | | | | | 5009.268-2016 |
| | | | | | Second method |
| Zinc | mg/100 g | 2.77 | 9.47 | 7.93 | GB/T |
| | | | | | 5009.268-2016 |
| | | | | | Second method |
| phosphorus | g/100 g | 476 | — | 596 (NY/ | GB/T |
| | | | | T1653- | 5009.268-2016 |
| | | | | 2008 Yao | Second method |
| | | | | Xingyu, 2017) | |
| Carbohydrate | g/100 g | 33.5 | — | — | By calculation ³ |
| Crude fiber | % | 8.1 | — | 15.5 (Yao | GB/T |
| | | | | Xingyu, 2017) | 5009.10-2003 |
| Total dietary fiber | g/100 g | 33.0 | — | — | GB 5009.88-2014 |
| fat | g/100 g | 0.82 | 4.46 | 3.16 | GB 5009.6-2016/ |
| | | | | | Second method |
| Moisture | g/100 g | 10.3 | — | 9.9 (NY/ | GB 5009.3-2016 |
| | | | | T1653- | First method |
| | | | | 2008 Yao | |
| | | | | Xingyu, 2017) | |
| Ash (by sample | g/100 g | 7.5 | 8.7 | 6.6 | GB 5009.3-2016 |
| mass) | | | | | First method |

Note 1:
The data of Pholiota adiposa and Pholiota microspora is from the master's paper of Jilin Agricultural University in 2015: the chemical composition and pharmacological activity of Pholiota adiposa - Wang Xiaoyan. The method for detecting amino acids is: GB/T 5009.124-2003. The rest is the same as the table. Individual missing data refer to the 2017 Kunming University of Science and Technology master's paper: the nutritional composition and chemical composition of Pholiota microspora-Yao Xingyu and has been marked in the form.
Note 2:
*is essential amino acid.
Note 3:
Carbohydrate (g/100 g) = 100 − (protein + fat + moisture + ash + dietary fiber)

From the nutrient composition of the new species of genus Pholiota in Table 3, it has all the essential amino acids and a high histidine content. Histidine is considered to be an essential amino acid for children in the field of nutrition. It can be synthesized in adulthood. It is an essential amino acid for uremic patients and is associated with various allergies and inflammation. Therefore, histidine is also an important class of amino acids. Compared with the nutritional components of the edible and medicinal fungi in “Medicinal and Edible Fungi”, the content of histidine is higher than 99% of the edible and medicinal fungi, and the content is high.

In addition, it can be seen from Table 3 that the crude polysaccharide content is also much higher than that of Pholiota microspora. Compared with these famous species such as Pholiota adiposa and Pholiota microspora, it is rich in potassium and potassium content is more than 2 times higher than that of Pholiota microspora.

In summary, the new species of genus Pholiota according to the present invention is a new species that has not been reported yet, and has not been studied yet, and has an effect of inhibiting Staphylococcus aureus, and is rich in nutrient content, and has high cultivation yield. It is a development prospects species with characteristics such as good traits.

The above is only a preferred embodiment of the present invention, but the scope of the present invention protection is not limited thereto, and for any person skilled in the art within the technical scope disclosed by the present invention, the technology according to the present invention equivalent replacements or modifications of the present invention and its concepts are intended to be included within the scope of the present invention.

1. A new species of genus Pholiota, Pholiota glutinosior, comprising HMGIM-W140054, and having an accession number of CCTCC NO: M 2019414.

2. An artificial cultivation method of the new species of genus Pholiota glutinosior of claim 1, comprising:
producing a first-class strain,
producing a second-class strain,
producing a third-class strain,
cultivating a culture having a cultivating medium in a cultivation bag,
wherein by weight percent, the cultivating medium comprises 28-32% cottonseed hulls, 56-58% wood chips, 8-12% bran, and 1-2% CaCO₃.

3. The artificial cultivation method according to claim 2, wherein by weight percent, the cultivating medium comprises 31% cottonseed hulls, 58% wood chips, 10% bran, 1% CaCO₃, and has a moisture content of 60% to 65%.

4. The artificial cultivation method according to claim 3, wherein the cultivation comprises:
transferring the third-class strain to the cultivating medium, keeping constant temperature at 25-26° C., shading the culture, keeping relative humidity at 60%-70%, and a cultivation management stage is entered when hyphae have overgrown the cultivation bag.

5. The artificial cultivation method according to claim 4, wherein the cultivation management stage comprises:
after the hyphae in the cultivation bag is full of the cultivating medium, the cultivation continues for 25 days after the shading to grow a mushroom with a cap, temperature is controlled at 18-20° C., and ventilation is increased to keep space carbon dioxide content below 1%; the relative humidity is adjusted to more than 90%;
after 14 days, the mushroom cap is removed and the hyphae begins to kink and form a pale yellow rice bran-like primordium; after the primordium grows to 0.5 cm, the temperature is kept at 18-20° C., relative humidity between 80-90%, exposure to 9 hours of light per day of light intensity 300-500 lx, and keeping carbon dioxide concentration in the air 350˜1500 ppm,
after 8 days, spraying the mushroom every day 1-2 times until the size of the mushroom fruit body is unchanged, and the fruit body cap no longer grows and begins to flatten, then harvesting the mushroom.

6. The artificial cultivation method according to claim 2, wherein production of the first-class strain comprises:
transferring isolated strains to a first-class strain culture medium, and placing the first-class strain culture medium in a constant temperature dark culture at 25° C.; when mycelium grows and bacteria have not grown, tip hyphae of the mycelium are picked up to obtain the first-class strain; or,
production of the second-class strain comprises:
transferring the first-class strain to a second-class strain culture medium, and placing the second-class strain culture medium in a constant temperature dark culture at 25° C., and the mycelium grows full of slopes to obtain the second-class strain; or,
production of the third-class strain comprises:
inoculating the second-class strain into a third-class strain culture medium by ensuring that the second-class strain culture medium is buried in the first-class strain culture medium and is placed in a constant temperature dark culture at 25° C. until the hyphae are full of the third-class strain culture medium, to obtain the third-class strain.

7. The artificial cultivation method according to claim 3, wherein production of the first-class strain comprises:
transferring isolated strains to a first-class strain culture medium, and placing the first-class strain culture medium in a constant temperature dark culture at 25° C.; when mycelium grows and bacteria have not grown, tip hyphae of the mycelium are picked up to obtain the first-class strain; or,
production of the second-class strain comprises:
transferring the first-class strain to a second-class strain culture medium, and placing the second-class strain culture medium in a constant temperature dark culture at 25° C., and the mycelium grows full of slopes to obtain the second-class strain; or,
production of the third-class strain comprises:
inoculating the second-class strain into a third-class strain culture medium by ensuring that the second-class strain culture medium is buried in the first-class strain culture medium, and is placed in a constant temperature dark culture at 25° C. until the hyphae are full of third-class strain culture medium, to obtain the third-class strain.

8. The artificial cultivation method according to claim 4, wherein production of the first-class strain comprises:
transferring isolated strains to a first-class strain culture medium, and placing the first-class strain culture medium in a constant temperature dark culture at 25° C.; when mycelium grows and bacteria have not grown, tip hyphae of the mycelium are picked up to obtain the first-class strain; or,
production of the second-class strain comprises:
transferring the first-class strain to a second-class strain culture medium, and placing the second-class strain culture medium in a constant temperature dark culture at 25° C., and the mycelium grows full of slopes to obtain the second-class strain; or,
production of the third-class strain comprises:
inoculating the second-class strain into a third-class strain culture medium by ensuring that the second-class strain culture medium is buried in the first-class strain culture medium, and is placed in a constant temperature dark culture at 25° C. until the hyphae are full of third-class strain culture medium, to obtain the third-class strain.

9. The artificial cultivation method according to claim 5, wherein production of the first-class strain comprises:
transferring isolated strains to a first-class strain culture medium, and placing the first-class strain culture medium in a constant temperature dark culture at 25° C.; when mycelium grows and bacteria have not grown, tip hyphae of the mycelium are picked up to obtain the first-class strain; or,
production of the second-class strain comprises:
transferring the first-class strain to a second-class strain culture medium, and placing the second-class strain culture medium in a constant temperature dark culture at 25° C., and the mycelium grows full of slopes to obtain the second-class strain; or,
production of the third-class strain comprises:
inoculating the second-class strain into a third-class strain culture medium by ensuring that the second-class strain culture medium is buried in the first-class strain culture medium, and is placed in a constant temperature dark culture at 25° C. until the hyphae are full of third-class strain culture medium, to obtain the third-class strain.

10. The artificial cultivation method according to claim 6, wherein the first-class strain culture medium is Bengal red medium comprising, by weight percent:
peptone 0.5%, glucose 1%, potassium dihydrogen phosphate 0.1%, magnesium sulfate (MgSO₄.7H₂O) 0.05%, agar 2%, 1/3000 Bangladesh Red solution 10%, chloramphenicol 0.01%, and the remainder is water; or,
the second-class strain culture medium is an enriched integrated potato dextrose agar, comprising, by weight percent:
20% potato, 20% glucose, 1% peptone, 2% agar, 0.3% potassium dihydrogen phosphate, 0.15% magnesium sulfate, 0.15% vitamin B1, and the remainder is water;
by weight percent, the third-class strain culture medium comprises: 98-99% sorghum and 1-2% calcium carbonate.

11. The artificial cultivation method according to claim 7, wherein the first-class strain culture medium is Bengal red medium comprising, by weight percent:
peptone 0.5%, glucose 1%, potassium dihydrogen phosphate 0.1%, magnesium sulfate (MgSO₄.7H₂O) 0.05%, agar 2%, 1/3000 Bangladesh Red solution 10%, chloramphenicol 0.01%, and the remainder is water; or,
the second-class strain culture medium is an enriched integrated potato dextrose agar, comprising, by weight percent:
20% potato, 20% glucose, 1% peptone, 2% agar, 0.3% potassium dihydrogen phosphate, 0.15% magnesium sulfate, 0.15% vitamin B1, and the remainder is water;
by weight percent, the third-class strain culture medium comprises: 98-99% sorghum and 1-2% calcium carbonate.

12. The artificial cultivation method according to claim 8, wherein the first-class strain culture medium is Bengal red medium comprising, by weight percent:
peptone 0.5%, glucose 1%, potassium dihydrogen phosphate 0.1%, magnesium sulfate (MgSO₄.7H₂O) 0.05%, agar 2%, 1/3000 Bangladesh Red solution 10%, chloramphenicol 0.01%, and the remainder is water; or,
the second-class strain culture medium is an enriched integrated potato dextrose agar, comprising, by weight percent:
20% potato, 20% glucose, 1% peptone, 2% agar, 0.3% potassium dihydrogen phosphate, 0.15% magnesium sulfate, 0.15% vitamin B1, and the remainder is water;
by weight percent, the third-class strain culture medium comprises: 98-99% sorghum and 1-2% calcium carbonate.

13. The artificial cultivation method according to claim 9, wherein the first-class strain culture medium is Bengal red medium comprising, by weight percent:
peptone 0.5%, glucose 1%, potassium dihydrogen phosphate 0.1%, magnesium sulfate (MgSO₄.7H₂O) 0.05%, agar 2%, 1/3000 Bangladesh Red solution 10%, chloramphenicol 0.01%, and the remainder is water; or,
the second-class strain culture medium is an enriched integrated potato dextrose agar, comprising, by weight percent:
20% potato, 20% glucose, 1% peptone, 2% agar, 0.3% potassium dihydrogen phosphate, 0.15% magnesium sulfate, 0.15% vitamin B1, and the remainder is water;
by weight percent, the third-class strain culture medium comprises: 98-99% sorghum and 1-2% calcium carbonate.

14. Use of the Pholiota glutinosior CCTCC NO: M 2019414 of claim 1 or an extract thereof for treating bacteria, wherein
the Pholiota glutinosior or the extract are used for preparation of the medicament for treating diseases caused by anti-bacteria or for preparation of a health supplement; the extract is preferably ethyl acetate extract; the bacteria is Staphylococcus, preferably Staphylococcus aureus;
the health supplement is rich in histidine, or crude polysaccharide, or potassium content.

15. The medicament for treating diseases caused by anti-bacteria of claim 14, comprising the Pholiota glutinosior CCTCC NO: M 2019414 or the extract thereof and a carrier.
